(19) United States
(12) Patent Application Publication
(10) Pub. No.: US 2021/0072182 A1
(43) Pub. Date: Mar. 11, 2021

(54) SENSOR DEVICE FOR THE PARALLEL DETERMINATION OF A CONCENTRATION OF SMALL MOLECULE SUBSTANCES AND OF A PH VALUE

(71) Applicant: SIEMENS AKTIENGESELLSCHAFT, München (DK)

(72) Inventors: Günter Fafilek, Bisamberg (AT); Martin Joksch, St.Andrä-Wördern (AT); Stefan Wibihal, Wien (AT); Johannes Österreicher, Perchtoldsdorf (AT)

(21) Appl. No.: 17/053,640

(22) PCT Filed: May 8, 2019
(86) PCT No.: PCT/EP2019/061825

§ 371 Date: Nov. 6, 2020

(30) Foreign Application Priority Data
May 9, 2018 (DE) 10 2018 207 275.2

(51) Int. Cl. G01N 27/48; G01N 27/416; G01N 27/30

(52) U.S. Cl. CPC G01N 27/48; G01N 27/302; G01N 27/4167

(57) ABSTRACT

Disclosed is a sensor device (100) for the parallel determination of a concentration of small molecule substances (e.g. ethanol, glucose, etc.) and of a pH value in a solution during the course of a biotechnological process using what is known as cyclic voltammetry. Said sensor device is constructed in the form of a rod electrode. The sensor device consists at least of two rod-shaped working electrodes (102, 103), for which different conductive materials are selected in such a way that distinguishable voltage/current profiles can be determined for the small molecule substances to be determined, and of a rod-shaped reference electrode (104). The working electrodes and the reference electrode are each embedded or melted in a tubular, insulating material. Furthermore, the sensor device comprises a counter electrode (105) in the form of a hollow cylinder, within which the working electrodes and the reference electrode are mounted. In addition, the sensor device has a sensor head part (101), which comprises at least one electronic component (107) for providing desired voltage profiles for cyclic voltammetry and for signal amplification, an analysis unit (108) for control and evaluation, and an interface (109) for data transmission.

### TECHNICAL AREA

The present invention relates to a sensor device for the parallel determination of small molecule substances—such as alcohols (methanol, ethanol, etc.) or other substances involved in cell metabolism (e.g. glucose, glutamine, lactate, lactose, acetate, etc.)—and of a pH value of a solution using so-called cyclic voltammetry. The concentration of the small molecule substances and the pH value are determined during the course of a biotechnological process.

### STATE OF THE ART

Microorganisms (e.g. bacteria, fungi, plants and/or animal cells) are often used in many biotechnological processes in the pharmaceutical industry and in the beverage industry. These are usually researched in detail and are then used, for example, to convert an organic substance—a so-called fermentation process—as part of its enzyme-catalyzed metabolism. Usually, the microorganisms are added to a bioreactor or fermenter with a nutrient solution in order, for example, to form desired substances (e.g. antibiotics, insulin, hyaluronic acid, alcohol, etc.) or to cultivate such microorganisms.

Biotechnological processes usually take place in a bioreactor or fermenter in which certain microorganisms or cells are cultivated or bred under optimal conditions, or in which the desired substances are produced by the microorganisms or cells, for example by means of a fermentation process. The solution in the bioreactor, which is also referred to as reactor broth, usually contains nutrients such as sugar or glucose, etc. for the microorganisms. These nutrients are then at least partially converted by the microorganisms into other, small molecule substances such as alcohols (e.g. ethanol), etc. or complex organic substances such as proteins. For a control of the respective biotechnological process in the bioreactor as well as for the quality of the respective product, however, knowledge of the respective process variables such as pH value, composition of the reactor broth, etc. is of crucial importance.

During a biotechnological process, the nutrients required for the microorganisms, such as sugar, glucose, etc., are usually made available by the nutrient solution or the reactor broth. However, the solution also absorbs the metabolic products of the microorganisms—i.e. the desired product—such as alcohols, etc. These metabolic products can have a toxic effect on the microorganisms, especially in high concentrations. Therefore, the knowledge of the alcohol content—in particular the content of ethanol and/or methanol—in the solution for the control of biotechnological processes and the quality of the respective product is of great importance. The pH value of the solution is also decisive for the course of the process and the quality of the product. Since, due to metabolic processes of the microorganisms during the biotechnological process, the solution can have a different pH value in the different phases, continuous control of the pH value is important in order to keep it as constant as possible at a value that is optimal for the respective production, for example by adding base. It is therefore important for process management and control of the course of the process to be able to determine the content of small molecule substances (e.g. methanol, ethanol, glucose, etc.) in the solution and the pH value of the solution.

For example, pH sensors are used to measure the pH of a solution. Potentiometric measurements can be carried out from these pH sensors on the basis of ion-selective electrodes, for example. For example, a device filled with a so-called buffer solution (e.g. a glass membrane ball) is immersed in the solution to be measured. Since, for example, hydrogen ions tend to accumulate in thin layers on the surface of the device, depending on the pH difference, a galvanic voltage is built up between the inside and outside of the device or the pH measuring sensor, which can be measured using two reference electrodes. The sensors can be designed as rod sensors for the laboratory area or as online sensors for the process industry. With sensors of this type, however, only the current pH value of the solution can be determined; other measuring or analysis devices and/or sensors are necessary for measuring the content of small molecule substances such as ethanol, glucose, etc.

A content of, for example, ethanol, glucose, etc. in a solution can be determined, for example, with the aid of an analysis device such as, for example, a gas chromatograph, a high-performance liquid chromatograph (HPLC), etc. In chemistry, chromatography is, for example, a process that enables a mixture of substances, such as a solution, to be separated by differently distributing its individual components between a stationary and a mobile phase. Gas chromatography can be used, for example, to separate a mixture into individual chemical components which can be vaporized in gaseous or undecomposed form. High-performance liquid chromatography is a liquid chromatography process in which substances (e.g. a sample of a solution) are not only broken down into components, but a concentration of these can also be determined. Alternatively, the content of small molecule substances such as ethanol, glucose, etc. can be determined by means of mass spectrometry or with the aid of a mass spectrometer.

However, the use of chromatography and mass spectrometry has the disadvantage that complex analytical devices are necessary for this, which are associated with relatively high costs. Furthermore, continuous sampling of the reactor broth or solution and evaluation of these samples is necessary for a parallel and current determination of the respective concentration of various small molecule substances in a reactor sample and a corresponding process control. Such a procedure is technically complex, cost-intensive and not suitable for what is known as online use (i.e. during the ongoing process, for example in the bioreactor). Samples must also be taken in a sterile manner to avoid contamination with other microorganisms.

Alternatively, optical spectroscopic methods such as near infrared spectroscopy (NIR), infrared spectroscopy (IR), etc. can also be used for the parallel determination of the content of small molecule substances in a solution. However, these are not infrequently either too imprecise or too complex to use. Furthermore, the content of small molecule substances such as ethanol, glucose, etc. can also be determined on the basis of an enzymatic reaction or by means of enzymatic tests, electrochemically, for example with the aid of biosensors. When using enzyme-based tests or biosensors for a parallel determination of the concentration of small molecule substances in a solution, there is the disadvantage that, for example, the enzymes are consumed during the respective measurement. The respective sensor must therefore be replaced after a certain number of measurements and can therefore only be used for a limited time. This excludes use as an online sensor. These sensors can only be operated as so-called atline sensors, for which a sampling system is necessary. A necessary exchange of such a sensor results in additional expenses and costs.

From the publication “Paixao, Thiago R. L. C et al: Amperometric determination of ethanol in beverages at copper electrodes in alkaline medium, Analytica Chimica Acta 472 (2002) 123-131, ” for example, a sensor with a copper electrode in an alkaline medium for measuring the ethanol content in beverages is known, in which, a principle of so called cyclovoltammetry is used. Cyclic voltammetry, which is also known as the triangular voltage method, is an analytical method with which, for example, various electrode processes can be examined. In cyclic voltammetry, cycles of rising and falling potential are applied to a working electrode in a solution, and a profile of a current between the working electrode and a counter electrode is recorded against a profile of the potential between the working electrode and a reference electrode. An electrochemically active substance is converted when the potential rises when the specific potential for this substance is reached, whereby a current rises. A concentration of this substance in the vicinity of the electrode decreases rapidly and with it the current. Another current only flows if the substance is replenished by diffusion. A measured peak in the profile of the current is then characteristic of the converted substance. For example, a concentration of the substance can be derived from the height of the peak in the current profile—for example, according to the so-called Randles-Sevcik equation, the height of the peak is proportional to the concentration of the substance. The disadvantage of the method or sensor described in the publication “Paixao, Thiago R. L. C et al:” is that only the concentration of a small molecule substance or of ethanol is determined. For biotechnological processes such as fermentation processes, however, it is necessary to determine several substance concentrations—e.g. of methanol, ethanol, etc.—in parallel.

The document DE 10 2013 202 003 A1 discloses a method and an arrangement for the direct and parallel determination of small molecule substances such as ethanol, methanol in a reactor broth, wherein so-called cyclic voltammetry is used to determine the concentration of the substances. The arrangement proposed in DE 10 2013 202 003 A1 for carrying out the method, however, has a relatively complex structure, which only needs to be charged with the reactor broth to be analyzed via a feed line, which is very expensive. Flexible handling in the laboratory or simple incorporation into a biotechnological system is not possible. Furthermore, the arrangement is only suitable for a parallel determination of alcohols. To determine the concentration of additional substances and/or the pH value, additional working electrodes or a corresponding sensor must be provided.

### PRESENTATION OF THE INVENTION

The invention is therefore based on the object of specifying a sensor device for the parallel determination of a concentration of small molecule substances and of a pH value using cyclic voltammetry, by means of which the disadvantages of the prior art are easily overcome and flexible and simple use is made possible.

This object is achieved by a sensor device of the type mentioned at the outset with the features according to the independent claim. Advantageous embodiments of the present invention are described in the dependent claims.

According to the invention, the object is achieved by a sensor device of the type mentioned at the outset, which is constructed in the form of a rod electrode. The sensor device according to the invention comprises at least two rod-shaped working electrodes and a rod-shaped reference electrode. Different conductive materials are selected for the working electrodes in such a way that distinguishable voltage and/or current profiles can be determined for the small molecule substances to be determined. Furthermore, like the reference electrode, the working electrode is embedded in an insulating material. The sensor device also has a counter electrode which is designed as a hollow cylinder and in which the working electrodes and the reference electrode are attached or bundled. Furthermore, the sensor device has a sensor head part which, for example, can also be designed in the shape of a cylinder. This sensor head part comprises at least one electronic component for providing desired voltage profiles for cyclic voltammetry and for signal amplification, an analysis unit for control and evaluation, and an interface for data transmission, for example to an external data processing system or a control and/or monitoring device.

The main aspect of the solution proposed according to the invention is that a sensor device that can be used flexibly is made available in a simple and inexpensive manner. With this sensor device, concentrations of several small molecule substances, in particular ethanol and glucose, can be determined in parallel and continuously, as well as the pH value during a biotechnological process in a solution, in a very simple manner and with little technical effort according to the principle of cyclic voltammetry. The respective electrodes which are used for the measurements have the great advantage over biosensors, for example, that no enzymes are used, which are consumed, and can therefore be used for a large number of measurements.

By using several—at least two—working electrodes made of different materials, different analytes or substances can ideally be determined in parallel using the same measuring principle. Since cyclic voltammetry can also be viewed as a combination of potentiometry (electrochemical analysis method based on an electrochemical potential) and amperometry (analysis method based on an electrochemically generated current flow), a pH value of the solution can be very easily derived as a side result when determining the concentration of the respective substances, especially ethanol and glucose. Due to the high polarization of the electrodes in cyclic voltammetry, the electrodes are also continuously cleaned, as a result of which the sensor device according to the invention is characterized by high stability. Furthermore, the sensor device can be used flexibly due to its rod-shaped structure and can be used very easily as a so-called online sensor—for example for ongoing measurements in a biotechnological plant.

It is advantageous if a first working electrode is made of palladium. With the help of this working electrode, it is very easy—as in the text “Gerstl, Matthias; Joksch, Martin, Fafilek, Guenter: The dissolution of palladium as a function glucose concentration in chloride containing solutions of acidic pH, Journal of Electroanalytical Chemistry 741 (2015) 1-7”—to determine a glucose concentration. A second working electrode is ideally made of platinum, a platinum alloy or another noble metal and is used, for example, to determine the ethanol concentration and the pH value. A silver/silver chloride electrode can be used as a reference electrode. Due to the different materials of the working electrodes, concentrations of e.g. glucose (e.g. with the first working electrode made of palladium) and ethanol (with the second working electrode made of e.g. platinum) as well as the pH value can be analyzed very easily using cyclic voltammetry. This makes use of the fact that, for example, a chemical conversion of an analyte depends not only on its electrochemical potential, but also on the catalytic effect of different electrode materials and on the three-dimensional structure of the respective molecule that is to be determined, whereby an attachment to the respective electrode is determined. In other words, depending on the electrode material, the different analytes deliver different current/voltage profiles with, for example, different current peaks, which are shaped differently in terms of position and shape and can be used to determine the content of, for example, ethanol and glucose in a solution.

For example, in order to be able to determine the concentrations of other small molecule substances important for cell metabolism and/or a process control, such as glutamine, lactate, acetate, glycerine, arabinose, lactose, etc., additional electrodes or working electrodes made of other noble metals can be used. The corresponding materials (e.g. copper (Cu), doped diamond, etc.), which for example have characteristic current/potential profiles and/or current peaks for the respective substance, must then also be selected for these working electrodes.

It is also advantageous if the working electrodes and the reference electrode are designed as microelectrodes. These have the advantage that they can be used, for example, in very small vessels and with even very small amounts of samples. A correspondingly optimal diameter is important for microelectrodes. For the sensor device according to the invention, this is, for example, in a range from 50 to 100 micrometers.

It is also advantageous if the counter electrode configured as a hollow cylinder is made from a non-corrosive, conductive material, in particular stainless steel. The counter electrode, designed as a stainless steel tube, serves to bundle and protect the working electrodes and the reference electrode. These are attached inside the counter electrode, for example by gluing. The sensor device is therefore particularly robust—especially if it is not installed in a system, but is designed, for example, as a hand-held device for laboratory operation.

Ideally, the working electrodes and the reference electrode are embedded in an insulating material. The insulating material used is advantageously glass or a glass tube in each case, into which the electrodes are each fused. The electrodes are electrically separated from one another by the insulating material or the glass and can nevertheless be attached in a very narrow space or within the counter-electrode designed as a hollow cylinder. The sensor device can be constructed in a handy and rod-shaped form for flexible use.

In a preferred development of the sensor device according to the invention, a detachably attached connecting element or flange is provided in the area of the counter electrode designed as a hollow cylinder. With the help of this flange, the sensor device can be connected to a vessel, for example a shake flask in a laboratory or a bioreactor vessel in a biotechnological plant. The connecting element can also be used, for example, to ensure a sterile sealing of the vessel.

In a preferred embodiment, the sensor device also has a battery module for a power supply in the sensor head part. The sensor device can thus be used autonomously—i.e. a connection to a power supply is not absolutely necessary.

A so-called multivariant analysis method, such as the so-called partial least square regression or PLS regression, can expediently be used for the evaluation of measured signal profiles or cyclic voltagrams by the analysis unit. The partial least square regression (PLS regression) is, for example, a statistical method with which a regression of independent so-called x variables on one or more so-called y variables is calculated. With the sensor device according to the invention, on the basis of the measured current and voltage profiles (cyclovoltagrams), for example, relationships and dependencies of voltage and current can be analyzed and the substance concentrations sought can be estimated. In order to be able to determine the desired measured variables or substance concentrations, it is necessary to import parameters into the sensor device depending on the matrix (composition) of the solution to be examined. These parameters can be obtained, for example, by calibration. While the sensor device is in operation, calibration can take place, for example, by means of so-called one-point calibration.

A microcontroller can ideally be used for the analysis unit, which controls the cyclovoltammetric measurements, digitizes the measured values and evaluates the measured signal profiles or cyclovoltagrams using multivariant analysis methods. Furthermore, by combining multivariate analysis methods (such as PLS regression) with several working electrodes made of different materials, it is possible, with little technical effort, to determine concentrations of several small molecule substances (e.g. ethanol and glucose) in parallel, which only differ slightly from an electrochemical point of view.

For flexible forwarding of the measurement data evaluated by the analysis unit to, for example, a higher-level control and/or monitoring system, the interface for data transmission can be designed either as a wired interface or as a wireless interface. In the case of a wired interface, for example, protocols used in the process industry can be used, such as the so-called Modbus protocol, etc. or a connection by means of a field bus, which is based on Ethernet, for example. For data transmission via a wireless interface, so-called near field communication or NFC, for example, can be used, which enables standardized, contactless exchange of data, for example.

Depending on the preferred area of use, the sensor device itself can either be designed as a hand-held device for a laboratory operation or installed in a plant for biotechnological processes. When used as a hand-held device in the laboratory, the sensor device can alternatively or additionally have a display unit for outputting and displaying results of the analysis unit to the interface for data transmission. The sensor device can advantageously be used autonomously in the laboratory for any test positions.

The sensor device can still be used in many applications, e.g. in the food and beverage industry as well as in the chemical or pharmaceutical sector and can be used, for example, both in production for process control (e.g. as a so-called online sensor installed in a plant) and in quality control (e.g. in a laboratory) in order to be able to check product properties, especially after transport and/or long storage. Since the sensor device provides stable measurements for a longer period of time and its structure can be used autonomously, the sensor device can also be used, for example, in the field of environmental analysis, in particular at, for example, poorly accessible measuring points.

### BRIEF DESCRIPTION OF THE DRAWING

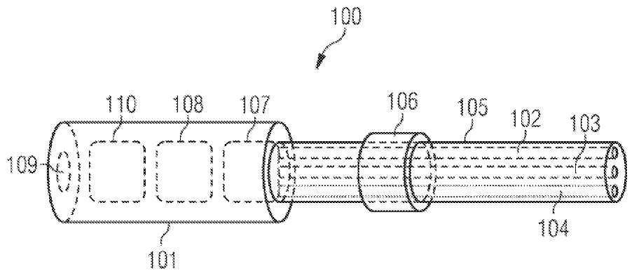

The invention is explained below using examples with reference to the attached FIGURE. FIG. 1 shows by way of example and schematically a sensor device for the direct and parallel determination of small molecular substances by means of cyclic voltammetry and of a pH value, which can be used during a biotechnological process.

### IMPLEMENTATION OF THE INVENTION

FIG. 1 shows by way of example and schematically the sensor device 100 according to the invention for the parallel determination of small molecule substances, in particular ethanol and glucose, as well as of a pH value in a solution using cyclic voltammetry. The sensor device 100 according to the invention is constructed in the form of a rod electrode and can be produced in at least two design variants. On the one hand, the sensor device 100 can be designed as a hand-held device for laboratory operation, i.e. as an autonomously operating sensor device 100. On the other hand, the sensor device 100 can be installed, for example, in a plant for biotechnological processes (e.g. production plant). That is to say, the sensor device 100 is integrated into the system in a fixed manner, for example by screwing or another method of fastening.

The sensor device 100 according to the invention has a sensor head part 101 in which the electronics required for measurement and data transmission are accommodated, as well as an electrode part. The electrode part comprises at least two rod-shaped working electrodes 102, 103, a rod-shaped reference electrode 104, and a counter electrode 105. The working electrodes 102, 103 and the reference electrode 104 are designed as microelectrodes, for example, which ideally have a diameter in a range from 50 to 100 μm. Furthermore, the working electrodes 102, 103 and the reference electrode 104 are each embedded in an insulating material. Glass or a glass tube, in which the respective electrode 102, 103, 104 is embedded or melted, can be used as the insulating material.

The working electrodes 102, 103 also consist of different, conductive materials, which are selected in such a way that, with these conductive materials, distinguishable current/voltage profiles can be determined for the small molecule substances to be determined. To determine a glucose concentration in the solution, a first working electrode 102 is made of palladium, for example. A second working electrode 103 is, for example, used to determine a pH of the solution and to determine an ethanol concentration in the platinum solution. Alternatively, platinum alloys or other noble metals can also be used for the second working electrode 103. The reference electrode 104 can be designed, for example, as a silver/silver chloride electrode.

The counter electrode 105 is designed as a hollow cylinder, in which the working electrodes 102, 103 embedded in insulating material or glass and the reference electrode 104 embedded in insulating material or glass are attached for bundling and protection. The working electrodes 102, 103 and reference electrode 104 can, for example, be glued to the counter electrode 105 designed as a hollow cylinder. The counter electrode 105 is made of a non-corroding, conductive material such as stainless steel, for example, so that cyclic voltammetric measurements are possible, but the counter electrode 105 is not attacked by the solution to be examined.

A detachable connecting element 106 or flange 106 can also be attached in the area of the counter electrode 105, which is designed as a hollow cylinder. This connecting element 106 is also designed in the shape of a hollow cylinder and can be used to connect to vessels in which the sensor device 100 is to be used.

The electronics required for the entire measurement or for cyclic voltammetry and for data transmission are located in the sensor head part 101, which can also be cylindrical. For this purpose, the sensor head part 101 comprises an electronic component 107, from which the voltages or voltage profiles necessary for cyclic voltammetry are provided and by which the signals or current/voltage profiles of the electrode part are amplified. Furthermore, the sensor head part 101 has an analysis unit 108, which can be designed as a microcontroller. The tasks of the analysis unit 108 are to control the cyclovoltammetric measurement, to digitize measured signal or current/voltage profiles and to carry out an analysis/evaluation of the measured values. This analysis or evaluation can take place, for example, by means of a so-called multivariant analysis method such as, for example, partial least square regression (PLS).

Furthermore, the analysis unit 108 can also control a data transmission via an interface 109 for data transmission, for example from the sensor head part 101 to a higher-level unit (for example a control or control unit of a plant, etc.). The interface 109 for data transmission can be designed, for example, as a wired interface, which supports standard protocols in the process industry (for example, Modbus protocol, Ethernet-based fieldbus). Alternatively, the interface 109 can also be designed, for example, as a wireless interface via which the evaluated measurement data is transmitted, for example by means of near-field communication or NFC.

The sensor head part 101 also includes a battery module 110. The electronic component 107 accommodated in the sensor head part 101 and the analysis unit 108 are supplied with energy by the battery module 110. Depending on the area of application (for example process control, ongoing analysis, quality control), the sensor device 100 can be designed as a hand-held device for laboratory operation or it can be installed in a plant for carrying out biotechnological processes.

In the embodiment of the sensor device 100 as a hand-held device for laboratory operation, the sensor head part 101 can also have a display unit as an alternative or in addition to the interface 109 for data transmission. The measured values determined with the sensor device 100, such as, for example, pH value, concentration of ethanol and glucose, etc., can be displayed and output directly on the sensor device 100 on the display unit.

For the determination of, for example, ethanol and glucose concentration as well as the pH value in a solution, the sensor device 100 with the electrode part is brought into contact with the solution to be analyzed, so that the working electrodes 102, 103, which consist of different materials (e.g. palladium and platinum), the reference electrode 104 and the counter electrode 105 are in contact with the solution. From the electronic component 107, to which the working electrodes 102, 103, the reference electrode 104 and the counter electrode 105 are connected, alternately selected potential profiles (e.g. triangular, etc.) are applied to the working electrodes 102, 103. In parallel with this, associated current profiles between the respective working electrodes 102, 103 and the counter electrode 105 as well as associated voltage profiles between the working electrodes 102, 103 and the reference electrode 104 are measured by the electronic component 107 for the applied potential profiles.

When measuring the corresponding, associated current profiles between the working electrodes 102, 103 and the counter electrode 105 and the corresponding, associated voltage profiles between the working electrodes 102, 103 and the reference electrode 104, the current profiles in particular are characteristic of a substance concentration to be determined in the solution to be analyzed. The voltage profiles measured between the working electrodes 102, 103 and the reference electrode 104 are largely predetermined by the selected potential profiles applied with the electronic component 107.

The electronic component 107 functions here, for example, as a so-called potentiostat, which in the simplest case can be used in electrochemistry as a precise DC voltage source or as a source for time-varying voltage profiles (e.g. triangle, etc.) or as a voltmeter or ammeter. The application of the potential profiles and the measurement of the respective associated current and voltage profiles between the electrodes 102, 103, 104, 105 are controlled and monitored by the analysis unit 108.

The measured current and voltage profiles or signal profiles are optionally amplified by the electronic component 107 and forwarded to the analysis unit 108. The measured signal profiles are digitized by the analysis unit 108 and evaluated, for example, using so-called multivariate analysis methods such as partial least square regression.

By evaluating the current and voltage profiles measured on working electrodes 102, 103, which are made of different materials—e.g. palladium and platinum—using multivariate analysis methods, a concentration of the small molecular substances to be analyzed (e.g. ethanol, glucose, etc.) can then be determined in parallel in the solution to be analyzed. For this purpose, for example, relationships between current and voltage profiles are estimated and, for example, the concentration of the respective small molecule substance such as ethanol or glucose is determined on the basis of current peaks that occur. As a side result of the determination of the content of small molecule substances such as glucose and ethanol by means of cyclic voltammetry in the solution to be analyzed, the pH value of this solution can also be determined.

### LIST OF REFERENCE SYMBOLS

100 Sensor device
101 Sensor head part
102 First working electrode
103 Second working electrode
104 Reference electrode
105 Counter electrode
106 Detachable connecting element (flange)
107 Electronic component for providing the voltage profiles for cyclic voltammetry and for signal amplification
108 Analysis unit for control and evaluation
109 Interface for data transmission
110 Battery module for a power supply

1.-12. (canceled)

13. A sensor device for the parallel determination of a concentration of small molecule substances and of a pH value during the course of a biotechnological process, the sensor device being constructed in the form of a rod electrode, the sensor device comprising:
two rod-shaped working electrodes, for which different, conductive materials are selected in such a way that distinguishable voltage and/or current profiles can be determined for the small molecule substances to be determined;
a rod-shaped reference electrode, wherein the working electrodes and the reference electrodes are embedded in an insulating material;
a counter electrode formed as a hollow cylinder, within which the working electrodes and the reference electrode are attached; and
a sensor head part which has at least one electronic component for providing desired voltage profiles for cyclic voltammetry and for signal amplification, an analysis unit for control and evaluation, and an interface for data transmission.

14. The sensor device of claim 13, wherein a first working electrode is made of palladium, a second working electrode is made of platinum, a platinum alloy or another noble metal, and the reference electrode is made of silver or silver chloride.

15. The sensor device of claim 13, wherein the working electrodes and the reference electrode are microelectrodes.

16. The sensor device of claim 13, wherein the counter electrode is configured as a hollow cylinder made from a non-corrosive, conductive material, in particular stainless steel.

17. The sensor device of claim 13, wherein glass is used as the insulating material for embedding the working electrodes and the reference electrodes.

18. The sensor device of claim 13, wherein a battery module for a power supply is also provided in the sensor head part.

19. The sensor device of claim 13, wherein a multivariant analysis method is used for the evaluation of measured signal profiles by the analysis unit.

20. The sensor device of claim 13, wherein the interface for data transmission is a wired or wireless interface.

21. The sensor device of claim 13, wherein the sensor device has a detachably attached connecting element in an area of the counter electrode being a hollow cylinder.

22. The sensor device of claim 13, wherein the sensor device is a hand-held device for laboratory operation.

23. The sensor device of claim 13, wherein the sensor head part comprises a display unit for outputting and displaying results of the analysis unit.

24. The sensor device of claim 13, wherein the sensor device is installed in a system for biotechnological processes.
